(19) United States
(12) Patent Application Publication
(10) Pub. No.: US 2026/0007854 A1
(43) Pub. Date: Jan. 8, 2026

(54) REUSABLE URINARY CATHETER PRODUCTS

(71) Applicant: Hollister Incorporated, Libertyville, IL (US)

(72) Inventors: Michael G. Murray, Ballina (IE); Horacio Montes de Oca, Ballina (IE); Malford E. Cullum, Grayslake, IL (US); Gary W. Inglese, Deerfield, IL (US); Rory Grogan, Dublin (IE)

(21) Appl. No.: 19/325,467

(22) Filed: Sep. 10, 2025

Related U.S. Application Data
(63) Continuation of application No. 17/617,822, filed on Dec. 9, 2021, now Pat. No. 12,440,644.
(60) Provisional application No. 62/866,339, filed on Jun. 25, 2019.

(51) Int. Cl. A61M 25/00; A61L 2/18

(52) U.S. Cl. CPC A61M 25/002; A61L 2/18; A61M 25/0045; A61L 2202/122; A61L 2202/18; A61L 2202/24; A61M 2025/0019; A61M 2025/0046; A61M 2025/0056

(57) ABSTRACT

Reusable catheter products are provided for storing, transporting, and sterilizing reusable urinary catheters. The products include a case comprising a proximal section, a middle section, and a distal section. The case includes a cavity configured to receive a catheter; the cavity includes a hydration fluid. The catheter comprises a distal end, a proximal end and a tubular section therebetween and includes a hydrophilic coating. When the catheter is inserted into the case, the hydration fluid sterilizes and lubricates the catheter.

This application is a continuation of U.S. patent application Ser. No. 17/617,822, Dec. 9, 2021, which is the National Stage Entry of International Application No. PCT/US2020/039190, filed Jun. 23, 2020, which claims the benefit of and priority to U.S. Provisional Application No. 62/866,339, filed Jun. 25, 2019, all of which are hereby incorporated herein by reference.

### FIELD OF THE DISCLOSURE

The present disclosure generally relates to urinary catheters. More particularly, the present disclosure relates to reusable urinary catheter products.

### BACKGROUND

Catheters are used to treat many different types of medical conditions and typically include an elongated shaft that is inserted into and through a passageway or lumen of the body. Catheters, and in particular intermittent catheters, are commonly used by those who suffer from various abnormalities of the urinary system, such as urinary incontinence. With the advent of intermittent catheters, individuals with urinary system abnormalities can self-insert and self-remove intermittent catheters several times a day.

Urinary catheters are frequently provided as disposable, single-use items. A user will remove the catheter from a package, use the catheter once, and then dispose of the catheter and the package. Reusable urinary catheters could, thus, be advantageous in reducing the amount of waste created by the use of disposable catheters, but there are various challenges associated with the use of reusable catheters (including storage, transport, and sterilization) that must be overcome before widespread acceptance and use of reusable catheters.

There is a need for reusable catheter products and methods of sterilizing the same.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, the reusable catheter product includes a catheter and a case. The case includes a distal section, a proximal section, and a middle section therebetween. The case also includes a cavity configured to receive the catheter. The reusable catheter includes a distal end and a proximal end. The reusable catheter includes a hydrophilic coating. The case also includes a hydration fluid contained within the cavity. The hydration fluid includes a sterilization medium. The product allows the catheter to be repeatedly inserted and removed from the case, and yields a sterile and re-usable catheter.

In another aspect, the reusable catheter product further includes the case having a flexible middle section, and rigid proximal and distal sections. The flexible middle section may have grooves and/or ridges. The case may also include at least one grip, and the distal section of the case may define at least one cutout configured to mate with a catheter cap. Moreover, the catheter further includes the catheter cap configured to mate with the cutout.

In another aspect, a method for sterilizing the reusable catheter product is disclosed. The method includes inserting the hydration fluid comprising the sterilization medium into the cavity and inserting the catheter into the case by pushing the proximal end of the catheter into the proximal section of the case.

### BRIEF DESCRIPTION OF THE DRAWINGS

FIG. 1 is a side view of a reusable catheter product.

FIG. 2 is a cross-sectional view of the reusable catheter product of FIG. 1, showing a catheter positioned in a case.

FIG. 3 is a cross-sectional view of the reusable catheter product of FIG. 1, showing the case without the catheter.

FIG. 4 is a perspective view of the reusable catheter product of FIG. 1, showing the case in a bent configuration.

FIG. 5 is a perspective view of a multiuse catheter contained within a package.

### DESCRIPTION

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

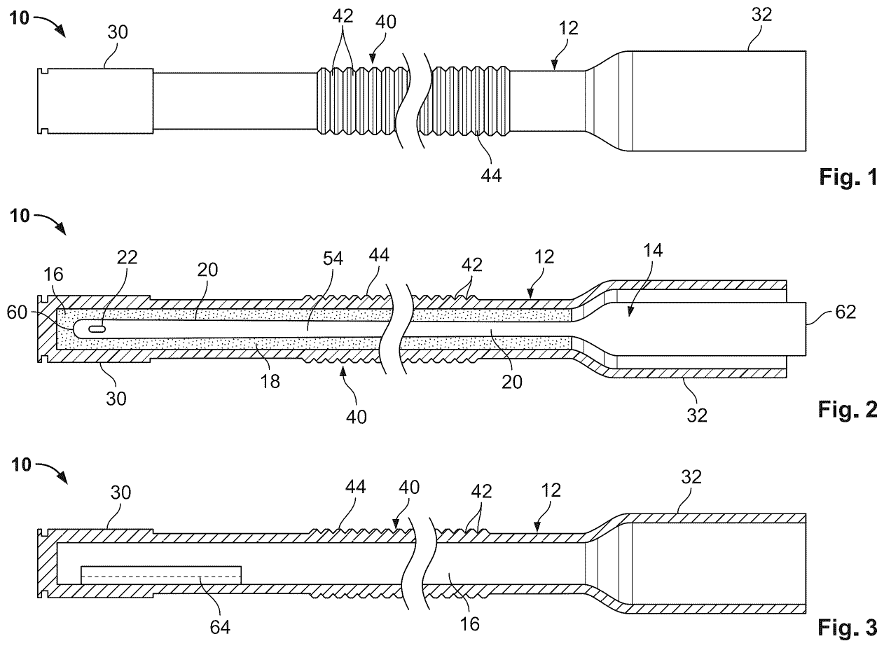

Reusable urinary catheter products according to the present disclosure and their individual components may be variously configured without departing from the scope of the present disclosure, but in one embodiment, a reusable urinary catheter product 10 is configured as shown in FIG. 1.

FIG. 1 shows a reusable urinary catheter product 10. The product 10 includes a storage and sterilization device, such as a case 12. Referring to FIGS. 2 and 3, the case 12 defines a cavity 16 configured to hold a hydration fluid 18. The case 12 includes a proximal section 30, a distal section 32, and a middle section 40 therebetween. The middle section 40 may be bendable or flexible. In the illustrated embodiment the middle section 40 includes grooves 42 and ridges 44 that assist in allowing the middle section 40 to bend. In one embodiment, the proximal section 30 and the distal section 32 are both comprised of rigid materials. The materials include but are not limited to PVC plastics and high density polyethylene (HDPE). Optionally, the middle section 40 comprises flexible material, including but not limited to elastomers.

The case 12 is configured to accommodate a multiuse urinary catheter 14. In an embodiment, the cavity 16 is configured to accept and store the urinary catheter 14. The catheter 14 includes a shaft 20 which has a hydrophilic coating thereon. The hydrophilic coating becomes lubricous when hydrated with a hydration fluid, which assists in easing insertion and withdrawal of the catheter 14 from the urethra.

The hydration fluid 18 comprises a sterilization medium for sterilizing the catheter 14 in between uses. The sterilization medium in the hydration fluid 18 contacts the catheter 14, when the catheter 14 is located within the cavity 16. The sterilization medium in the hydration fluid 18 can include any suitable antiseptic component such as iodine, oligodynamic metals, etc. In one embodiment, the hydration fluid 18 may include a water-based solution of a complex of polyvinyl pyrrolidone (PVP) and iodine. The hydration fluid 18 is capable of activating or refreshing the hydrophilic coating of the urinary catheter 14, and capable of sterilizing the catheter 14 when the coating contacts the hydration fluid 18. In one embodiment, when a user inserts the catheter 14 into the case 12, the catheter 14 may be immersed in the hydration fluid 18, which sterilizes and hydrates the hydrophilic coating of the catheter 14. In another embodiment, the catheter 14 may not be immersed in the hydration fluid 18 but the fluid 18 may slosh around within the case 12 due to shaking and/or natural movement of the user.

In an embodiment, the hydration fluid 18 comprises a water based solution of chlorhexidine (e.g., dihydrochloride, diacetate, and digluconate).

In another embodiment the sterilization medium of the hydration fluid 18 comprises FimH inhibitors that hinder bacterial adhesion to cells, tissues and synthetic material surfaces, thus preventing bacterial colonization and proliferation. The FimH inhibitors include, but are not limited to, mannose based molecules. Among these mannose based molecules are alkyl alfa-D-mannopyranosides (e.g. heptyl alfa-D-mannopyranoside) and aryl alfa-D-mannopyranosides, having different degrees of relative inhibitory potency. In an embodiment, the sterilization medium of the hydration fluid 18 includes antibacterial materials. These antibacterial materials include peptidomimetic antimicrobials (synthetic peptides that attack the prokaryotic membrane and destroy it). Examples of these are: Lys-Leu or klotho (KL) peptide known as (KLAKLAK)2, D-(KLAKLAK)2, brilacidin (PMX-30063) and POL7080.

In one embodiment the sterilization medium of the hydration fluid 18 comprises antibacterial materials including aminoglycosides and derivatives (bactericidal antibiotics tolerant to enzymatic inactivation). Examples of these include but are not limited to Plazomicin.

In another embodiment the sterilization medium of the hydration fluid 18 comprises nanoparticles with bactericidal effects. Examples of these include, but are not limited to, zinc oxide and copper oxide.

In another embodiment the sterilization medium of the hydration fluid 18 comprises oxazolidinones capable of inhibiting bacterial protein synthesis. These oxazolidinones include, but are not limited to, Tedizolid, Solithromycin, cethromycin, Omadacycline, fusidic acid, GSK1322322, Radezolid, Tedizolid S and Eravacycline.

In another embodiment the sterilization medium of the hydration fluid 18 comprises antibacterial proteins that inactivate bacterial DNA. These anti-bacterial proteins include, but are not limited to, SASPject technology. In another embodiment the sterilization medium of the hydration fluid 18 comprises a water solution with honey. In one embodiment the sterilization medium of the hydration fluid 18 comprises glucose oxidase.

In addition to the hydration fluid 18 having features which may sterilize the product 10, the catheter 14 and/or case 12 may also have features that facilitate sterilization. For example, an antimicrobial compound may be incorporated into the material of the catheter 14 and/or case 12. The antimicrobial compound may include, but is not limited to, zinc oxide, zinc sulphates, and zero valence silver.

In one embodiment zinc oxide is incorporated into the material of the catheter 14 and/or case 12. For instance, zinc oxide (ZnO) powder may be compounded using a twin screw extruder with the polymer material of the case, which may be a thermoplastic elastomer (TPE). This ZnO compound is then extruded to produce a case 12 and/or catheter 14 loaded with antimicrobial ZnO.

In one embodiment, when making the catheter 14, a polymer loaded with ZnO is formed into a catheter tube. The tube is tipped and a funnel is attached to the catheter 14. The catheter 14 is subsequently coated with a hydrophilic coating such as a polyvinylpyrrolidone (PVP) based coating. Once the catheter 14 is coated, at least one catheter “eye” 22 is cut into the tube (FIG. 2). Thus, the catheter 14 has an outer portion with a hydrophilic coating and an inner portion including a polymer-ZnO composite. Due to the antibacterial properties of the ZnO-polymer composite the inner surface will resist bacterial colonization. Similarly, the case 12 may be made from a loaded polymer, such as TPE, which inhibits bacterial colonization in the cavity 16 of the case 12. Furthermore, the outer surface of the catheter 14 and the inner surface of the case 12 may be UV irradiated to kill bacteria.

In another embodiment the hydrophilic coating of the catheter 14 is loaded with ZnO to provide an antibacterial hydrophilic surface. Alternatively, the catheter 14 and/or coating, comprises zinc sulphates (ZnSO4) and zero valence silver.

In another embodiment, the catheter 14 includes a silicone based coude tip/straight tip. The catheter 14 also may include zero valence silver attached to its exterior and interior walls. When metallic silver is deposited inside and outside of the catheter 14, the catheter 14 may remain bacteria free and be useable for a years-long time period.

In one embodiment, the case 12 includes a wall that is lined with silver and the case12 is filled with water. The silver lining helps kill bacteria both inside and outside the catheter 14.

In one embodiment, the case 12 includes a pouch that is lined with silver. The pouch is filled with water and is configured to hold the catheter 14. The silver lining helps kill bacteria both inside and outside of the catheter 14.

In one embodiment the hydration fluid 18 may include water containing a surfactant to loosen biofilm from the catheter 14.

In one embodiment the case 12 includes an agitator to agitate the hydration fluid 18 during sterilization of the catheter 14.

In one embodiment the catheter 14 is placed in a catheter pack capable of irradiating the catheter with antibacterial UV light 64 for sterilization (illustrated in FIG. 3). Optionally, the hydration fluid 18 may include a hydrophilic polymer, such as PVP. When the hydration fluid 18 contacts the hydrophilic coating, the hydrophilic polymer also contacts the coating. During exposure to UV light, the hydrophilic polymer of the hydration fluid 18 crosslinks with the hydrophilic coating so as to refresh the hydrophilic coating with additional polymer chains.

FIG. 2 is a cross sectional view of an embodiment of the product 10, showing the catheter 14 inside the case 12. As shown in FIG. 2, the case 12 is configured to accommodate the catheter 14. When a user inserts the catheter 14 inside the case 12, the hydration fluid 18 inside the case 12 sterilizes the catheter 14, allowing for multiple safe and sterile uses of the catheter 14. Moreover, the catheter eye 22 is visible at the proximal end 60 of the catheter 14, near the proximal section 30 of the case 12. The catheter eye 22 is configured to receive the hydration fluid 18. The catheter eye 22 allows the hydration fluid 18 to travel inside the catheter 14 so that the hydration fluid 18 sterilizes the inside of the catheter 14.

FIGS. 2 and 3 show the case 12 in a straightened configuration. The straightened configuration allows the catheter 14 to be fully inserted into the case 12. When in the straightened configuration, the flexible middle section 40 is planar and aligned with both the proximal section 30 and the distal section 32 of the case 12. Optionally, the case 12 may include a UV light 64 that may be used to sterilize the product 10 and refresh the hydrophilic coating when the hydration fluid 18 includes a hydrophilic polymer.

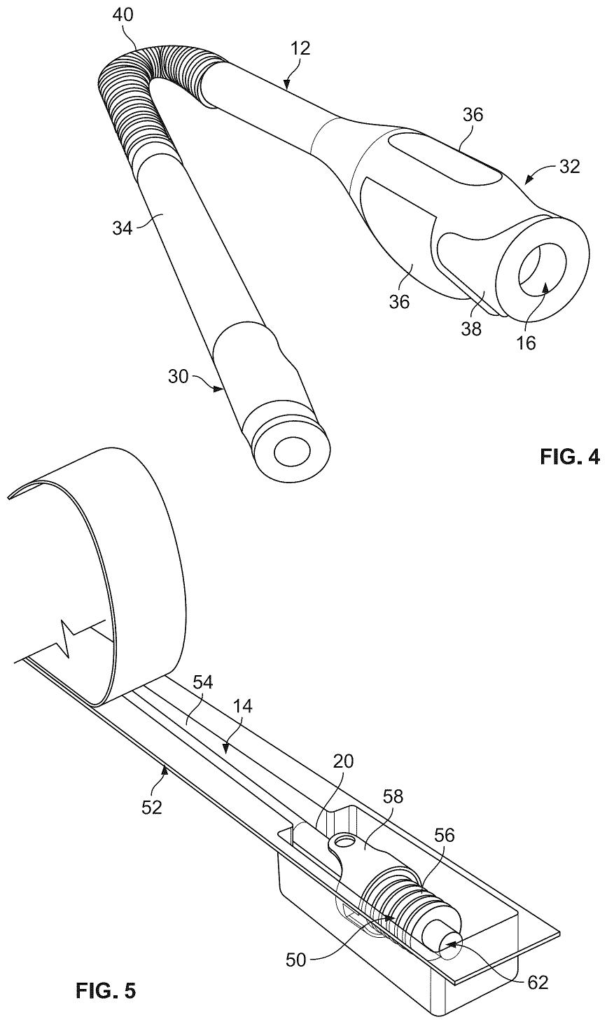

FIG. 4 shows an embodiment of the case 12 in a bent configuration.

The bent configuration allows a user to more easily store and carry the case 12. In the bent configuration the middle section 40 is flexed so that the distal section 32 and the proximal section 30 sit approximately opposed to each other. In the embodiment shown, the middle section 40 is bent at an acute angle. However, the flexible material comprising the middle section 40 allows for a range of angles; a user may choose the angle he or she prefers for added convenience.

FIG. 4 also shows grips 34, 36 attached to the outside of the case 12. The case 12 includes a lower grip 34 that surrounds the lower portion of the case 12. The lower grip 34 comprises a smooth elastomeric material, such as rubber. In the embodiment shown in FIG. 4, the lower grip 34 extends from the bottom of the middle section 40 to approximately ⅔ of the way to the proximal section 30. FIG. 4 also shows an upper grip 36. The upper grip 36 shown in FIG. 4 comprises a partially grooved elastomeric material (for example, rubber) and/or plastic. The upper grip 36 at least partially surrounds the upper portion of the case 12. In FIG. 4 the upper grip 36 extends from the distal section 32 to approximately the halfway point between the distal section 32 and the top of the middle section 40. The grips 34, 36 make it easier for the user to grasp and hold the product 10.

Furthermore, the distal section 32 comprises a cutout 38 configured to accommodate a cap 50 on the distal end 62 of the catheter 14 (the cap is shown as 50 in FIG. 5). The cap 50 and the cutout 38 fit together to help keep the catheter 14 in place while inserted into the case 12. This cap 50 and cutout 38 coupling may help keep the catheter 14 stable for traveling, sterilization, and/or for when the case 12 is in the bent configuration with the catheter 14 inside.

Additionally, FIG. 4 shows an embodiment wherein the distal section 32 has a wider section at the upper most portion of the case 12 that tapers distally from the bottom of the upper grip 36. The wider section of the upper portion of the case 12 extends from the bottom of the cutout 38 to the bottom of the upper grip 36. The narrower section of the upper portion extends from the bottom of the upper grip 36 to the top of the middle section 40. The case 12, the cavity 16, and the tapered upper portion may be configured to accommodate the shape of the catheter 14.

Referring to FIG. 5, the catheter 14 may be provided to a user in a disposable package 52. The catheter 14 comprises a proximal end 60 and a distal end 62 with a tubular section 54 therebetween. The tubular section 54 may comprise a shaft 20. The reusable catheter 14 may be used multiple times and then may be discarded for a new catheter 14. The catheter 14 shown in FIG. 5 also comprises a removable cap 50 configured to mate with the cutout 38 (shown in FIG. 4). The cap 50 includes a lower section 58 configured to engage the cutout 38 on the case 12.

As shown in FIG. 5, the removable cap 50 surrounds the distal end 62 of the catheter 14 and comprises a substantially rigid polymeric material including but not limited to plastic. The cap 50 closes off the distal end 62 of the catheter 14 so that when it is stored in the case 12, hydration fluid 18 does not leak from the distal end 62 of the catheter 14. Moreover, the cap 50 shown includes a portion with at least one groove 56. The at least one groove 56 spans the circumference of the cap 50.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A reusable urinary catheter product, comprising:
a case defining a cavity;
a reusable catheter including a catheter shaft having a distal end portion and a proximal end, the catheter shaft being located within the cavity, the catheter shaft including a hydrophilic coating thereon, the catheter being configured to be repeatedly inserted into and removed from the case; characterized in:
a hydration fluid within the cavity, the hydration fluid comprising a sterilization medium and polyvinyl pyrrolidone (PVP), wherein the hydration fluid hydrates the hydrophilic coating and sterilizes the catheter; and
the case comprising a sterilizing light source.

2. The product of claim 1 wherein the hydration fluid further comprises iodine.

3. The product of claim 1 wherein the middle section of the case is flexible and may be configured to bend, forming at least one angle.

4. The product of claim 1 wherein the middle section comprises grooves and ridges.

5. The product of claim 1 wherein the case includes at least one grip.

6. The product of claim 1 wherein the distal section of the case includes at least one cutout, wherein at least one cutout is configured to mate with the distal end portion of the catheter.

7. The product of claim 1 wherein the hydration fluid comprises at least one of: an anti-septic lubricant, a water-based solution of chlorhexidine, an oil-based lubricating agent, FimH inhibitors, antibacterial materials, nanoparticles with bactericidal effects, oxazolidinones, antibacterial proteins, a water solution with honey, glucose oxidase, a water solution with silver, or a water lubricant with a surfactant configured to loosen a biofilm from the catheter.

8. The product of claim 1, wherein one or more of zinc oxide, zinc sulphates, or zero valence silver is incorporated into the case and/or the catheter.

9. The product of claim 1, wherein the sterilizing light source comprises UV light.

10. The product of claim 1, wherein the case includes an agitator to agitate the hydration fluid during sterilization of the catheter.

11. The product of claim 1, wherein the case comprises at least one sliver lined wall.

12. The product of claim 7, wherein the FimH inhibitors include mannose based molecules, preferably, alkyl alfa-D-mannopyranosides, or aryl alfa-Dmannopyranosides.

13. The product of claim 7, wherein the antibacterial materials include peptidomimetic antimicrobials, preferably, (KLAKLAK)2, D-(KLAKLAK)2, brilacidin (PMX-30063), or POL7080.
